(19) United States
(12) Patent Application Publication
(10) Pub. No.: US 2026/0117039 A1
(43) Pub. Date: Apr. 30, 2026

(54) UPCYCLING WASTE POLYSTYRENE TO ADIPIC ACID THROUGH A HYBRID CHEMICAL AND BIOLOGICAL PROCESS

(71) Applicant: Alliance for Energy Innovation LLC, Golden, CO (US)

(72) Inventors: Gregg Tyler BECKHAM, Golden, CO (US); Hyunjin MOON, Golden, CO (US); Mikhail Olegovich KONEV, Lakewood, CO (US)

(21) Appl. No.: 19/373,502

(22) Filed: Oct. 29, 2025

Related U.S. Application Data
(60) Provisional application No. 63/713,394, filed on Oct. 29, 2024.

(51) Int. Cl. C08J 11/16; C07C 51/265; C07C 51/31; C07C 51/36

(52) U.S. Cl. CPC C08J 11/16; C07C 51/265; C07C 51/31; C07C 51/36; C08J 2325/06

(57) ABSTRACT

Disclosed herein are methods for oxidative catalytic depolymerization of polystyrene (PS) can produce benzoic acid, but the annual consumption of benzoic acid is about 40 times lower than PS. For this catalytic oxidation method to be a viable means to manage PS waste, benzoic acid should be converted to higher-volume chemicals. We demonstrate a hybrid chemical and biological process that uses PS as feedstock for production of adipic acid, a high-volume co-monomer for nylon 6,6 via benzoic acid. Mn/Br co-catalyzed autoxidation of PS to benzoic acid proceeds with a yield of up to 94% in a solvent mixture of benzoic acid and water. The PS-derived benzoic acid undergoes bioconversion at near-quantitative yield to muconic acid, which is readily converted to adipic acid through catalytic hydrogenation.

### CROSS-REFERENCE TO RELATED APPLICATIONS

This application claims priority under 35 U.S.C. § 119 to U.S. provisional patent application No. 63/713,394 filed on 29Oct.2024, the contents of which are hereby incorporated in their entirety.

### CONTRACTUAL ORIGIN

The United States Government has rights in this invention under Contract No. DE-AC36-08GO28308 between the United States Department of Energy and Alliance for Sustainable Energy, LLC, the Manager and Operator of the National Renewable Energy Laboratory.

### BACKGROUND

Polystyrene (PS) is widely used in multiple commercial and consumer applications, including packaging, construction, and electronics that benefit from its durability, low weight, and insulating properties. In 2022, the global annual consumption of PS was 18 million metric tons (MMT), accounting for 6% of total global plastic production. According to data from the US EPA, the lack of post-consumer recycling infrastructure for PS resulted in 91% of PS in the U.S. (2.8 MMT) being landfilled and 8.5% incinerated in 2019, though some amount of recycling may not be captured in these numbers. Consequently, new methods for PS mechanical recycling and chemical recycling to monomers are gaining significant attention. Advances have been made in the densification of expanded polystyrene (EPS) foam using solvent-based and mechanical techniques, which facilitate subsequent mechanical recycling.

Efforts to convert PS into other aromatic chemicals are the focus of growing attention and have led to multiple oxidation methods that convert PS into benzoic acid. Recent studies have primarily featured photochemical methods, which generate benzoic acid in yields up to 73%. Over 20 years ago, others demonstrated that PS can be converted to benzoic acid with an 88% yield via catalytic autoxidation with a Co/Mn/Br/Zr co-catalyst in acetic acid, conditions that closely resemble the commercial Mid-Century (MC) process for conversion of p-xylene to terephthalic acid. These conditions, however, lead to additional process costs arising from the need to recover acetic acid by distillation. In addition, the insolubility of PS in acetic acid at autoxidation temperature hinders the oxidative reactivity of the polymer. Use of benzoic acid as a solvent could address both limitations. Benzoic acid is the product of PS autoxidation, and its use as a solvent for the process could overcome the limitations of acetic acid and simplify the overall process. Benzoic acid readily dissolves PS and would enable the oxidation reaction to occur in a homogeneous solution, potentially improving the autoxidation kinetics and/or reducing the solvent demand by allowing for higher substrate loading during the reaction.

The appeal of improving the conversion of PS into benzoic acid is offset by the mismatch in the commercial market for benzoic acid (0.56 MMT/yr) and to the annual volume of PS waste (3.1 MMT in the U.S. in 2019). This mismatch means that benzoic acid will need to be converted into one or more other products with higher market demand. Microbial aromatic catabolism provides an efficient strategy to convert benzoic acid into muconic acid, which may be readily converted into existing commodity chemicals, including adipic acid, terephthalic acid, adiponitrile, acrylic acid, and caprolactam, or used directly in performance-advantaged bioproducts. Hydrogenation of muconic acid to produce adipic acid, one of the two co-monomers in nylon 6,6, is particularly appealing. Adipic acid has a growing market demand (3 MMT globally in 2022) that is well-matched to the volume of PS waste. Adipic acid is currently produced via benzene hydrogenation to cyclohexane, cyclohexane autoxidation to KA oil (a mixture of cyclohexanone and cyclohexanol), and oxidation of KA oil with HNO₃ with Cu/V co-catalysts (FIG. 1A). The KA oil oxidation step generates substantial greenhouse gases (GHGs: N₂O, CO, and CO₂) and would be ideally replaced with a more sustainable chemical pathway for adipic acid production.

### SUMMARY

In an aspect, disclosed herein is a method for making adipic acid from polystyrene comprising autoxidation of polystyrene in a solution of benzoic acid to generate benzoic acid wherein the benzoic acid is metabolized by a non-naturally occurring microbe to make muconic acid; and wherein the muconic acid is hydrogenated to make adipic acid.

Other objects, advantages, and novel features of the present invention will become apparent from the following detailed description of the invention when considered in conjunction with the accompanying drawings.

### BRIEF DESCRIPTION OF THE DRAWINGS

FIGS. 1A and 1B depict two synthetic routes to adipic acid. FIG. 1(A) depicts the primary production route to adipic acid from petrochemically-derived benzene. FIG. 1(B) depicts an embodiment of a route in this work from PS waste to benzoic acid, bioconversion to muconic acid, and hydrogenation to adipic acid.

FIGS. 2A, 2B, 2C, 2D, 2E and 2F depict reaction optimization for PS autoxidation. FIG. 2(A) depicts a comparison of benzoic acid and acetic acid as solvents for PS oxidation. Reaction conditions: PS (Mₙ 91.0 kg/mol, Ð=2.9) 0.45 g, benzoic acid 1.0 g or acetic acid 10 g, Co/Mn/Br co-catalyst (3.6, 3.6, and 4.0 wt %, relative to the PS mass), 165° C., 2 h, O₂ 7 bar/N₂ 53 bar at RT. FIG. 2(B) depicts benzoic acid yields when using various catalyst systems. For Co/Mn/Br co-catalyst, a loading of 3.6, 3.6, and 4.0 wt % (relative to the PS mass) is used, respectively. For other catalyst systems, the loadings of Co(OAc)₂, Mn(OAc)₂, and NaBr are 7.1, 7.1, and 4.0 wt %, respectively. FIG. 2(C) depicts the effect of NaBr loading from 0 to 6.2 wt % at 7.1 wt % Mn(OAc)₂. FIG. 2(D) depicts the influence of the PS mass loading in benzoic acid on the reaction performance. FIG. 2(E) depicts the reaction profile when using benzoic acid and H₂O as co-solvents. The error bar represents the error from triplicates, and the bar in 2(C) and 2(D) represents the range of yields obtained from duplicates. FIG. 2(F) depicts images of post-consumer PS used in autoxidation reactions and the corresponding yields of benzoic acid. Reaction conditions: Post-consumer PS 0.45 g, benzoic acid 1.0 g with water 1.0 g, Mn(OAc)₂ 7.1 wt %, NaBr 1.0 wt %, 165° C., 4 h, O₂ 7 bar/N₂ 53 bar at RT.

FIGS. 3A, 3B and 3C depict bioconversion of EPS-derived benzoate to muconate. FIG. 3(A) depicts a metabolic pathway from benzoate to cis,cis-muconate in P. putida KT2440-CJ074 (genotype: P. putida KT2440 ΔcatRBC::P_{tac}:catA1 ΔpcaHG). Shake flask cultivations with P. putida KT2440-CJ074 in M9 minimal medium supplemented with glucose and either FIG. 3(B) commercial benzoic acid (solid lines) or FIG. 3(C) substrate from the autoxidation of post-consumer EPS (dashed lines). Error bars represent the standard deviation across three biological replicates. FIG. 3(D) depicts bioreactor cultivations with P. putida KT2440-CJ074 fed with commercial benzoate (solid lines) and substrate from the autoxidation of EPS (dashed lines) using a dissolved oxygen (DO)-stat fed-batch strategy. Bacterial growth was tracked as optical density at 600 nm (OD₆₀₀). Data from singlet experiments are reported.

FIG. 4 depicts a simplified process flow diagram for the oxidation of PS to benzoic acid, biological conversion of benzoic acid to muconic acid, and muconic acid hydrogenation to adipic acid. Abbreviations: Prod. —productivity, MA—muconic acid.

### DETAILED DESCRIPTION

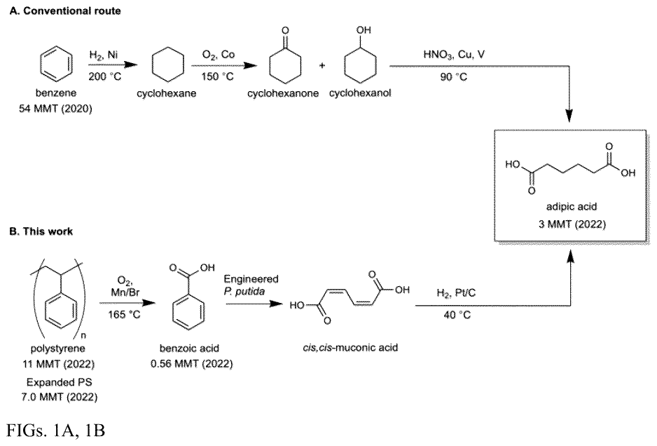

Disclosed herein are methods and compositions of matter for the conversion of PS to adipic acid via a hybrid chemical and biological process, involving PS autoxidation to benzoic acid, bioconversion of benzoic acid to muconic acid, and hydrogenation of muconic acid to adipic acid (FIG. 1B). The PS autoxidation conditions are optimized to enable use of a benzoic acid and water co-solvent system instead of acetic acid and to simplify the previously reported Co/Mn/Zr/Br catalyst system to a composition that only consists of Mn/Br. Benzoic acid obtained from PS autoxidation undergoes biological conversion to cis,cis-muconic acid using the engineered microbe Pseudomonas putida KT2440-CJ074. Finally, adipic acid is obtained via Pt/C-catalyzed hydrogenation of muconic acid. The results highlight the merits of this approach, in addition to illuminating further opportunities for improvement of these pathways to produce benzoic, muconic, and adipic acids as products.

### PS Deconstruction to Benzoic Acid

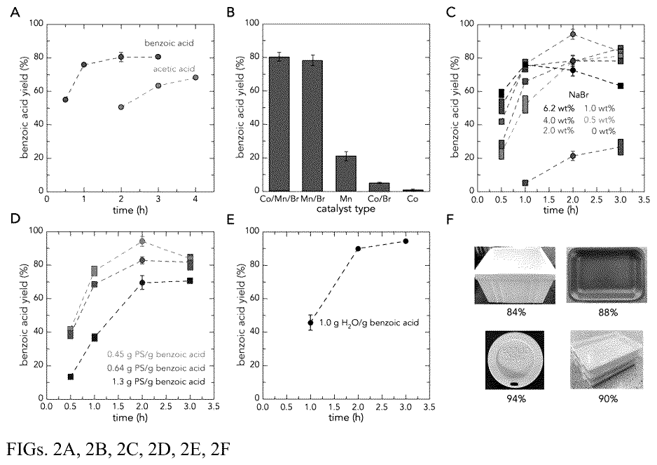

Autoxidation of PS to benzoic acid has been previously demonstrated in acetic acid using a Co/Mn/Br/Zr catalyst system and under related MC-like process conditions. To examine the feasibility of using benzoic acid as a reaction solvent, we compared PS autoxidation in acetic acid and benzoic acid under identical conditions: 0.45 g of PS beads (Mₙ 91.0 kg/mol, Ð=2.9), 165° C., 7 bar partial O₂ pressure, and catalyst loadings of 3.6 wt % Co(OAc)₂, 3.6 wt % Mn(OAc)₂, and 4.0 wt % NaBr relative to the PS mass (resembling loadings used in previous PS autoxidation studies). Initial experiments using 1.0 g of each solvent revealed that PS has poor solubility in acetic acid, even at elevated temperature. The polymer beads aggregated, and no formation of benzoic acid was observed during the reaction in acetic acid. PS was still insoluble in 10 g of acetic acid, but this condition enabled benzoic acid production. Thus, we used this condition to perform the control experiment. PS showed good solubility in benzoic acid at 165° C. and did not require adjustment of the solvent quantity. A comparison of these two experiments (FIG. 2A) showed that the use of benzoic acid as the solvent increases both the rate and yield of PS-derived benzoic acid, relative to the reaction performed in acetic acid. The yield of benzoic acid is defined by the molar ratio of PS-derived benzoic acid relative to the aromatic monomer units present in PS. The amounts of benzoic acid were quantified with ultra-high performance liquid chromatography with diode array detection (UHPLC-DAD) (for the reaction in benzoic acid, the solvent used in the reaction was subtracted from the total benzoic acid present at the end of the reaction.) The lower rate in acetic acid is likely affected by the poor solubility of PS in acetic acid at 165° C.

The promising initial result obtained with benzoic acid as a solvent provided the basis for further optimization of the catalyst composition. A Mn/Br co-catalyst (7.1 wt % and 4.0 wt %, respectively) led to a yield of 78% in benzoic acid solvent (FIG. 2B), comparable to that obtained with the Co/Mn/Br co-catalyst system. The reaction time courses with the Co/Mn/Br and Mn/Br co-catalysts showed similar trends from 0.5 to 3 h, indicating that Co is not an essential co-catalyst in this autoxidation reaction. Use of a Co/Br catalyst system resulted in only a 5% yield, and individual Co and Mn catalysts lacking bromide also performed poorly (FIG. 2B). Variation of the Mn(OAc)₂ loading at 4.0 wt % NaBr showed that 7.1 wt % exhibited the optimal rate and product yield of benzoic acid.

The NaBr loading has a substantial impact on benzoic acid production, with data showing a trade-off between rate and yield. Higher yields of benzoic acid were observed at higher NaBr loadings at 0.5 h, but deviations from this trend were evident at longer times. The best yield of 94% benzoic acid was observed with 2.0 wt % loading of NaBr at 2 h (FIG. 2C), with a decrease in yield at longer times. Overall, the trends in FIG. 2C indicate that NaBr accelerates the formation of benzoic acid but can also contribute to its decomposition. The oxidative sensitivity of benzoic acid has also been observed in PS oxidation by nitric acid, and control experiments show that benzoic acid decomposes in the absence of PS under the present conditions. Overall, these data show that a Mn/Br co-catalyst system with benzoic acid as the solvent enables improved rate and yield of PS depolymerization (94% yield after 2 h at 165° C.) relative to previously reported conditions with a Co/Mn/Br/Zr co-catalyst system in acetic acid (88% yield after 5 h at 180° C.). The results also enable a significant reduction in solvent demand by supporting reaction performance at 0.45 g PS/g benzoic acid rather than the 0.036 g PS/g acetic acid using previously existing methods.

Efforts to increase the PS mass loading beyond 0.45 g PS/g benzoic acid led to suboptimal outcomes (FIG. 2D), and no benzoic acid was formed in the absence of added solvent. Analysis of reaction products by gel permeation chromatography (GPC) reinforced these observations. Under the optimized reaction conditions (0.45 g PS/g benzoic acid, 7.1 wt % Mn(OAc)₂, 2.0 wt % NaBr; cf. orange trace in FIG. 2D), the product mixture features a broad molar mass distribution at 0.5 h, but rapidly converges to products with molar masses less than 590 g/mol by 1 h. GPC analysis of the reaction with a 1.3 g PS/g benzoic acid mass loading showed high molecular weight products were still present at 1 h.

PS Deconstruction with H₂O Co-Solvent to Improve the Process

The TEA presented below revealed that the exothermic reaction under high PS loadings in benzoic acid could lead to challenges with heat management at scale. A potential solution to this problem is the use of a co-solvent with lower boiling point. Water is the ideal option. Previous studies of MC oxidation of methylbenzene derivatives indicated that the reaction can proceed with 5-20 wt % water in acetic acid, albeit at a slower rate. Similar observations were made in the present system upon addition of 1.0 g of water to the standard reaction conditions, containing 0.45 g PS/1.0 g benzoic acid. A decrease in rate was observed, but a 94% yield of benzoic acid yield was still obtained at 3 h (FIG. 2E). Due to the process advantages of this co-solvent system (elaborated below), these conditions were adopted for the oxidative deconstruction of post-consumer PS samples. Post-consumer PS contains various additives that could affect autoxidation reaction outcomes, and thus we evaluated the depolymerization of post-consumer PS products, including white EPS, black EPS, a white coffee cup lid, and a yellow container, all of which were characterized thoroughly. For EPS autoxidation using a co-solvent system consisting of an equal ratio of benzoic acid and H₂O, a benzoic acid yield of 84% was obtained at 4 h (FIG. 2F), showing an increasing trend in benzoic acid yield over time. Under the same reaction condition for EPS, the autoxidation of black EPS, the white coffee cup lid, and the yellow container resulted in yields of 88%, 94%, and 90%, respectively. Thus, the autoxidation using benzoic acid as a solvent can efficiently deconstruct PS products as well, achieving high benzoic acid yields despite the presence of unknown additives. Since the purity of each post-consumer PS sample and the substrate sizes were different, the optimal reaction time to achieve the highest yield would also vary.

Benzoic Acid Recovery from EPS Oxidation

To isolate benzoic acid for bioconversion to muconic acid, the reaction mixture obtained from white EPS oxidation (cf. FIG. 2F, 84% crude yield) was subjected to vacuum distillation at 200° C. for 0.5 h. This process led to high recovery of the benzoic acid product (82% isolated yield). The product purity was measured by differential scanning calorimetry (DSC) melting point analysis and found to be 99.84%, similar to the purity of commercially available benzoic acid (99.89%,). Recrystallization of this material resulted in an improved purity of 99.98%. Elemental analysis with inductively coupled plasma optical emission spectroscopy (ICP-OES) and mass spectroscopy (ICP-MS) showed negligible Mn content (below the detection limit) and a Br content of 0.004 wt %, which is sufficiently low to have no impact on the bioprocess.

### Bioconversion of EPS-Derived Benzoic Acid to Muconate

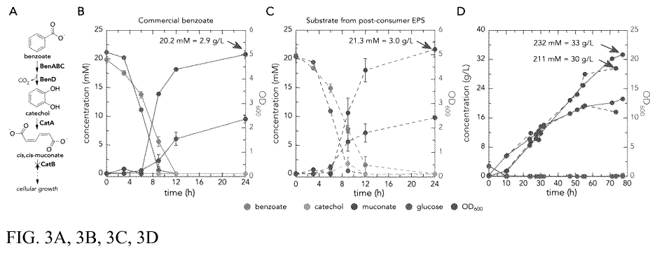

The purified benzoic acid was converted to muconate using an engineered strain P. putida KT2440-CJ074. In this strain, strong, constitutive, and chromosomal overexpression of the gene encoding CatA-I, a catechol 1,2-dioxygenase, enables conversion of benzoate-derived catechol to muconate that subsequently accumulates due to catB deletion (FIG. 3A). In shake flask experiments, P. putida KT2440-CJ074 showed similar growth profiles on either 20 mM of commercially available benzoate or substrate from EPS autoxidation, and when glucose was provided as a carbon and energy source (FIG. 3B, 3C). Transient catechol accumulation was higher in cultivations with EPS-derived substrate (1.8±1.1 mM) but quantitative molar conversion of benzoate to muconate was achieved on both substrates.

After confirming the successful bioconversion of deconstructed EPS to muconate in flasks, we scaled up the process to 0.5 L bioreactors. As a control, we simultaneously tested the conversion of pure benzoate under the same cultivation conditions to ascertain whether the accumulation of any potential toxic by-product(s) from the EPS-derived stream would affect the bioprocess. A dissolved oxygen (DO)-stat fed-batch strategy was used in this experiment to intermittently feed benzoate and glucose in small doses. This feeding strategy is generally effective for maximizing titers and yields while preventing substrate accumulation, particularly when (1) substrates are toxic and (2) a continuous feeding rate has not been established. However, this approach inherently limits productivity. Bacterial growth and muconate production followed the same values and trends for both commercial and EPS-derived benzoates, achieving ca. 30 g/L (232 and 211 mM, respectively) after 80 h (solid and dashed green lines, FIG. 3D), which confirms that recovered EPS-derived benzoate lacks toxic impurities that might inhibit the bioprocess in the tested conditions. Muconate productivity was 0.4 g/L/h from EPS, with molar yields of ca. 96% in both cases. Together these data show that EPS-derived substrate lack impurities that might inhibit the bioprocess in the tested conditions.

### Hydrogenation of Muconic Acid to Adipic Acid

Muconate produced in the bioreactors from EPS was separated and purified using activated carbon treatment, as described in our previous report⁴³. The purified muconic acid was used to produce adipic acid through hydrogenation with Pt/C (5 wt %) at 40° C. and 20 bar H₂. 150 mg of muconic acid in 15 g of ethanol was converted to adipic acid, showing a quantitative yield within 7.5 min, similar to results previously obtained. After the removal of ethanol, the purity of adipic acid, as measured by DSC, was 99.64 confirming that adipic acid of high purity can be obtained.

### Embodiments of TEA and LCA for PS Deconstruction and Conversion

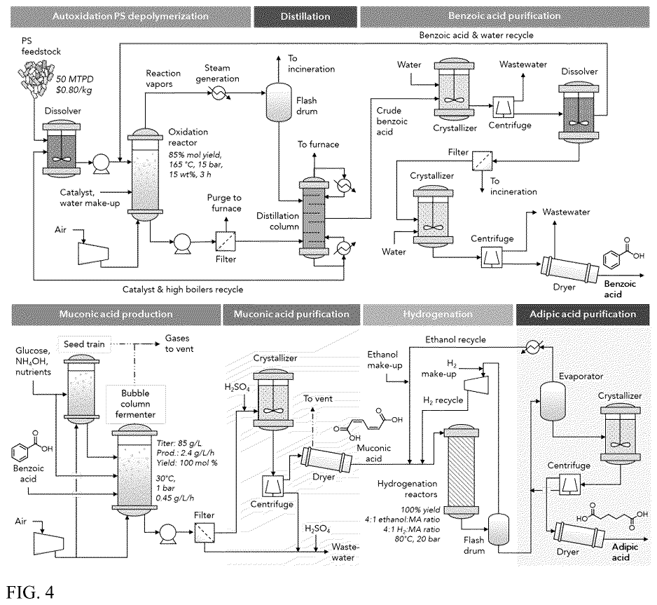

With the successful chemical and biological conversion of PS to adipic acid at the bench scale, an economic and environmental analysis was conducted to understand how the technology could perform at industrial scale. Conceptual process models for PS oxidation to benzoic acid and its successive conversion to adipic acid were developed in Aspen Plus V14, and TEA and LCA were conducted using the resulting material and energy balances. A simplified process flow diagram is shown in FIG. 4. The two processes of PS conversion to benzoic acid and benzoic acid conversion to adipic acid were considered to occur in co-located facilities allowing for detailed evaluation of each process. Importantly, the results for the co-located plants are nearly identical to those that would result from a single integrated process. The PS oxidation plant was assumed to have a feedstock capacity of 50 metric tons per day (MTPD), representing less than 1% of the total EPS and PS consumed within the U.S. in 20185. The feedstock capacity for the benzoic acid plant was also taken to be 50 MTPD as this is the amount of benzoic acid produced from the PS oxidation plant under base case conditions.

For the PS oxidation process, the feedstock was taken to be densified EPS sourced from a preprocessor as compressed blocks and delivered at a price of $0.80/kg. The feedstock was further assumed to consist of 95 wt % PS content with 5 wt % contaminants such as PP, PE, ash, and water. The oxidation process begins by dissolving the PS feedstock in a benzoic acid melt which is then pumped into the reactor alongside water in a 1:1 water to benzoic acid weight ratio. The reactor operates at 165° C. and 15 bar, with the water allowing for heat management in the continuous system via evaporative cooling. Mn(OAc)₂ and NaBr are present at 7 wt % and 2 wt % relative to PS, and air is sparged at a 4.5:1 mass ratio relative to PS. The benzoic acid yield is taken as 85% of theoretical for a 3 h residence time at a solid loading of 15 wt % (defined as the fraction of PS feedstock to the total stream mass). As the reaction proceeds, the solution partially vaporizes and is sent to a partial condenser thereby removing heat from the reaction and generating process steam. The gaseous fraction is separated and sent to incineration whereas the liquid fraction is continuously drawn off and filtered to separate any solid impurities or by-products from the reaction. The filtrate, containing benzoic acid and solubilized catalyst, is sent to a distillation column to produce crude benzoic acid and a concentrated stream of catalyst and other heavy boilers which is sent to incineration. The fly ash from incineration containing metal oxides from the catalysts is recovered and dissolved in acetic acid for reuse in the reaction with an assumed 95% recovery. The crude benzoic acid from distillation is combined with water and cooled to produce benzoic acid crystals, which are then recrystallized in water to produce the purified benzoic acid product. A fraction (27%) of the purified benzoic acid is recycled to the oxidation step as solvent make-up and the rest is sent for further purification.

For the conversion of benzoic acid, the purified product from oxidation is combined with water and fed to a set of bubble column reactors alongside corn-derived glucose and diammonium phosphate as a source of nitrogen. The reactors are inoculated with a cell culture from a seed train and the benzoic acid is converted to muconic acid with a productivity of 2.4 g/L/h and a titer of 85 g/L, as reported in Corynebacterium glutamicum by others. As noted above, the bioconversion results shown in the current study demonstrated that the purified benzoic acid from EPS does not inhibit P. putida CJ074 relative to commercial benzoic acid. However, we did not optimize the bioprocess for titer and rate despite opportunities to do so—for instance, using constant feeding instead of feeding via DO-stat mode could substantially increase productivity. Others have describes process optimization in a strain that is also likely more tolerant to higher muconic acid titers than P. putida. Hence we use their bioprocess performance as optimistic metrics for the process modeling efforts. Air is sparged into the bioreactors to facilitate the aerobic conversion and NH₄OH is added to maintain a neutral pH. After bioconversion, the muconate product is filtered to remove cell biomass and other solids, then acidified with H₂SO₄ in a crystallizer unit to produce crystalline muconic acid. The muconic acid is dried and mixed with ethanol in a 4:1 molar ratio, then sent to a packed bed reactor where it is hydrogenated over a 1 wt % Pt/Al₂O₃ catalyst using a 4:1 H₂ to muconic acid molar ratio at 20 bar and 85° C. H₂ is removed from the reactor product stream and passed through a pressure swing adsorption unit before being recycled; the remaining liquid fraction of the reactor product is flashed to recover and recycle the ethanol. The concentrated adipic acid stream is then crystallized at 15° C. and dried to produce the final adipic acid product.

### METHODS

### Chemicals

Manganese (II) acetate (Mn(OAc)₂, 98%), cobalt (II) acetate (Co(OAc)₂, 99.99%), sodium bromide (NaBr, ≥99%), benzoic acid (≥99.5%), benzoic acid-2,3,4,5,6-d₅ (≥99 atom % D), chloroform-d (99.8 atom % D), acetone-d₆ (99.9 atom % D), dimethyl sulfoxide (≥99.9%), tetrahydrofuran (THF, ≥99.9%), PS (280 kDa, Lot #: MKCL4619), cis,cis-muconic acid (≥97%), adipic acid (99%), and Pt/C (5 wt %, Lot #: MKBN5556V) were purchased from Sigma Aldrich. Toluene (≥99.8%) was purchased from VWR Chemical. Ethanol (200 proof) was obtained from Pharmco (Greenfield Global). Benzoic acid used for analytical standards was obtained from Acros Organics. Ultra-zero compressed air and ultra-high purity compressed nitrogen gas were purchased from Matheson.

### Catalytic Autoxidation of PS

All reactions were performed on Series 5,000 Multiple Reactor Parr Instruments, which provides for six 75 mL (total volume) batch reactors. Titanium reactors were used since the surface of stainless-steel reactor was corroded by NaBr at the reaction conditions. For a typical experiment, catalysts (Mn(Oac)₂, Co(Oac)₂, NaBr), benzoic acid, PS, and magnetic stir bar (length 10 mm, diameter 3 mm, PTFE) were added in a titanium reactor. Reactors were purged with N₂ twice before leak testing at 80 bar. Upon passing leak testing, the reactors were charged with ultra-zero compressed air to 33 bar and further diluted with N₂ to 60 bar. The reactors were heated to the desired reaction temperature for the target reaction time. The reaction times reported in this study includes a 30-min duration necessary to reach the desired temperatures.

After reaction completion, reactors were cooled down to ca. 20° C. in 30 min, then depressurized. All products (benzoic acid, residual PS, catalysts) in the headspace and reactor vessel were dissolved in THF and collected in a volumetric flask (25 mL). The solution in the flasks was diluted with THF for analysis. For the product solution containing cobalt ions, phthalic acid was added to the HPLC sample to achieve a mole ratio of phthalic acid to cobalt of 50:1, to inhibit the interaction between cobalt ions and benzoic acid.

Construction of P. putida KT2440-CJ074

The plasmid pMFL22 was used to delete the catRBC genes and replace them with the tac promoter upstream of catA in the genome of P. putida KT2440 (ATCC 47054) as previously described⁵⁶. The pcaHG genes were then deleted using pCJ011 as previously described, yielding KT2440-CJ074.

### Bacterial Strains, Media, and Shake-Flask Cultivations

The plasmid pMFL22 was used to delete the catRBC genes and replace them with the tac promoter upstream catA in the genome of P. putida KT2440 (ATCC 47054) and) as previously described. ThepcaHG genes were then deleted using pCJ011 as previously described, yielding KT2440-CJ074. Cultivations were performed in LB (Sigma-Aldrich #L3022) or M9 minimal medium (6.78 g/L Na₂HPO₄, 3 g/L KH₂PO₄, 0.5 NaCl, 1 g/L NH₄Cl, 100 mM CaCl₂, and 18 mM FeSO₄) supplemented with 20 mM glucose, 20 mM benzoate, or substrate from the autoxidation of EPS. A 100 mM benzoate stock was prepared by solubilizing benzoic acid (Sigma-Aldrich #242381) in water by pH adjusting to 7.0 with 1 M NaOH. A 100 mM benzoate stock was prepared from deconstructed EPS by assuming the weight was 100% benzoic acid and solubilizing the substrate in water via pH adjusting to 7.0 with 1 M NaOH. Both commercial benzoate and deconstructed EPS was provided to cultures at the same volumetric ratio (20% v/v). Glucose (Fisher Chemical #D16) was added from a 2.77 M stock prepared in water. All stock solutions were sterilized by 0.2 μm vacuum filtration prior to addition to the media. P. putida KT2440-CJ074 was revived from a glycerol stock by inoculating into Millers LB and cultivating at 30° C., 225 rpm for 12 h. Cell pellets were washed in M9 salts, inoculated into 30 mL media as described above to an OD₆₀₀ of 0.13-0.15 in 125 mL baffled metal-capped flasks, and cultivated at 30° C., 225 rpm. Glucose was fed to 20 mM every 24 h. OD₆₀₀ was measured with 50 μL culture, and samples were harvested for analyte quantitation by removing 0.5-1 mL, centrifuging at ≥15,000×g, passing through a 0.2 μm syringe unit, and storing in amber silanized vials at −20° C. until analysis.

### Bioreactor Conversion of Depolymerized EPS to Muconate

The bioconversion of the EPS-derived stream generated via catalytic autoxidation to muconic acid was performed by P. putida KT2440-CJ074 using a dissolved oxygen (DO)-stat fed-batch strategy and a modified protocol from previous work. In addition, we conducted a parallel experiment with commercial benzoate (sodium benzoate; B420; Sigma Aldrich) to identify potential differences compared to the EPS-derived stream. For the seed of both bioreactor campaigns, a scrape of a frozen glycerol stock was used to inoculate 25 mL of LB Miller media in independent 125 mL baffled flasks. The cultures were incubated 16.5 h at 30° C. at 225 rpm. Then, the cells were centrifuged at 5000 g for 8 min, the supernatant was removed, and the cells were resuspended in 5 mL of modified M9 medium (see below). The resuspended cells were used to inoculate the bioreactors to an initial OD₆₀₀ of 0.2. For the bioreactor experiments, OD₆₀₀ was measured using a nanodrop (Thermo Scientific).

For the bioreactor experiment, P. putida CJ074 was cultivated in 0.5 L BioStat-Q Plus bioreactors (Sartorius Stedim Biotech) containing 200 mL of modified M9 medium with 2.7 g/L (15 mM) of glucose. The modified M9 medium also contains 13.56 g/L, Na₂HPO₄, 6 g/L, KH₂PO₄, 1 g/L, NaCl, 2 g/L, (NH₄)₂SO₄, 2 mL/L of 1M MgSO₄ solution, 0.2 mL/L of 1 M CaCl₂) solution, 2 mL/L of 5 g/L FeSO₄ₓ7H₂O solution, and 1 mL/L antifoam 204 (A6426; Sigma Aldrich, St. Louis, MO). The cultivations were maintained at 30° C., 7.0 pH using 4 M NaOH, and sparged with air at 1.2 vvm. The initial agitation in the batch phase was set at 350 rpm. After 4 h, 1.6 mL of a filter sterilized (0.2 μm; Nalgene PES) 0.25 M solution of sodium benzoate (pH 7.5) was added to reach a concentration of 2 mM in the bioreactor. Once the saturation of oxygen reached 30%, the agitation was maintained at 30% using a DO cascade with a 5% dead band. When the glucose was depleted, the DO increased to 75%, at which point the fed-batch phase was initiated. The feed contained 1 M of benzoic acid, either from commercially-available sodium benzoate (144.11 g/L) or from the depolymerized EPS (122.12 g/L), 100 g/L (0.56 M) glucose, 13.5 g/L (0.10 M) ammonium sulfate, and 3.8 mL/L of antifoam 204, all adjusted to a pH of 7.5 with NaOH (10 M) and filter sterilized (0.2 μm; Nalgene PES). The feed was dosed for 30 seconds at a rate of 0.4 mL/min to target 1 mM benzoic acid concentration in the bioreactor in each pulse. The feed was automatically added every time the DO reached 75%. The agitation was manually adjusted to maintain a minimum DO of 10%. The experiment ended when all the feed—a total of 85.5 mL and 60 mL from the commercial benzoate and EPS-derived stream, respectively—was consumed. The spent broth from the EPS-derived stream was then centrifuged at 20,000 g and 30 min, filtered through 0.2 μm pore-size membranes (597-4520; Thermo Fisher) and used for further downstream separations and catalytic upgrading.

### Muconic Acid Purification

The purification of muconic acid obtained from the bioprocess was conducted by following the method previously described.

### Hydrogenation of Muconic Acid

20 mg of Pt/C (5 wt %), 150 mg of purified muconic acid, and 15 g of ethanol were added sequentially in a stainless-steel Parr reactor (75 mL). The reactor was flushed with H₂ three times and pressurized with H₂ at 20 bar while stirring at 600 rpm. Since hydrogenation occurs even at room temperature, heating began immediately after pressurizing with H₂ to 20 bar, which was marked as the reaction start time. The reaction temperature increased from RT to 40° C. after 30 min.

### Benzoic Acid, Benzaldehyde, and Muconic Acid Analysis

Benzoic acid, benzaldehyde, and cis,cis-muconic acid were analyzed using UHPLC-DAD as described previously⁶⁴ with the addition of benzoic acid, benzaldehyde and cis,cis-muconic acid as analytes. Calibration standards were used with an R² coefficient of 0.995 or better. Calibration verification standards for each analyte were analyzed every 12-24 samples to ensure the integrity of the initial calibration.

### Quantitation of Adipic Acid.

Adipic acid was quantified using the ultra-high performance liquid chromatography coupled with tandem mass spectrometry (UHPLC-MS/MS) method and parameters described previously.

### Gel Permeation Chromatography (GPC) Analysis

Molar mass analysis of PS and analysis of PS deconstruction products were performed using gel permeation chromatography (GPC) coupled with multi-angle light scattering (MALS) using an Infinity 111260 HPLC system from Agilent. The HPLC stack is equipped with a degasser, vial sampler, and heated column compartment with one guard column and three PLgel 5 μm MIXED-C 300×7.5 mm (purchased from Agilent) heated to 40° C. The mobile phase is HPLC-grade THF stabilized with butylated hydroxytoluene purchased from Spectrum Chemical running at a flow rate of 1 mL/min. The detectors used for GPC are a Wyatt Technology Optilab differential refractometer (dRI) and a Wyatt Technology miniDAWN light scattering detector (MALS). After the autoxidation reactions, THF was used to collect all products. Solid samples were prepared after removing THF by rotary evaporation (50° C., 250 mbar, further decreased to 180 mbar as the solvent amount decreases), followed by drying in a vacuum oven (50° C., 180 mbar). The solids were dissolved in THF at ˜20 mg/mL and filtered through a 0.2 μm PTFE syringe filter. Untreated PS samples were prepared in THF at a concentration of ˜5 mg/mL. A narrow molar mass distribution PS standard was used to normalize and align the detectors. A dn/dc value of 0.184 is used for PS samples. GPC traces of PS standards (Tosoh Biosciences, PStQuick kit C and D) were obtained to compare the retention time of PS standards with that of residual oxidized PS.

### NMR Spectroscopy

The ¹H NMR spectra of PS samples were recorded on a Bruker Ascend™ 300 MHz spectrometer. Chemical shifts were reported with respect to internal solvent (CDCl₃, δ(¹H) 7.26 ppm). DMSO was used to determine the purity of the PS substrates. The ¹H NMR spectra of muconic acid were measured using a Bruker Avance III HD 400 MHz spectrometer, with toluene as an external standard for determining the purity of muconic acid. The spectra were analyzed using MestReNova (v14.2.0, Mestrelab Research S.L.).

### Vacuum Distillation and Recrystallization

The separation of benzoic acid from the Mn/Br catalysts and residual PS was performed using a Kugelrohr apparatus (Büchi glass oven B-585) consisting of a four-bulb setup connected to a vacuum system (18 mbar). The first bulb contained 500 mg of products, and the temperature was increased to 200° C. It takes ca. 9 min to reach 200° C. from RT. The system was maintained at this temperature for 30 min to recover benzoic acid. For the recrystallization of benzoic acid, approximately 2.5 g of the distilled benzoic acid was combined with 50 g of H₂O, and the mixture was heated to 110° C. to dissolve the benzoic acid. The aqueous solution was then cooled to 3° C. in an ice bath, and filtration yielded purified benzoic acid.

### Differential Scanning Calorimetry (DSC)

DSC measurements for post-consumer PS samples were performed on a TA Discovery X3 Differential Scanning Calorimeter Instrument (TA Instruments). Samples of 3-10 mg were placed in hermetically sealed aluminum pans (DSC Consumables—Part #—DSC84012). DSC measurements were taken at a heating and cooling rate of 10° C./min from 0 to 160° C. The results were reported for the second heating/cooling cycle. DSC measurements were also conducted for benzoic acid and adipic acid samples to obtain purity values. A Discovery DSC 25 (TA Instruments) with an attached refrigerated cooling system −90° C. accessory (TA Instruments) was used to collect data. The heating rate was set to 0.5° C./min and sample size is roughly 1.7 mg. The data sampling interval was set to 1 sec/point. TA Instruments T-zero hermetically sealed pans were used (TA Instruments—Part #—900793.901). Purity was determined by analysis of the melting point following ASTM method E928 in the TA Instruments TRIOS software.

### Thermogravimetric Analysis (TGA)

TGA experiments were performed using a Discovery Series TGA 5500 (TA Instruments). Samples of 3-15 mg were loaded onto platinum pans for analysis. During analysis, the sample was purged with either nitrogen or air at a flow rate of 25 mL/min, with experiments conducted separately for each gas. The sample was heated at a rate of 20° C./min to a final temperature of 700° C. TA Instruments Trios Software was used for data analysis.

### ICP-OES

Manganese concentration was measured using an Agilent 5100 ICP-OES instrument configured with an SPS4 autosampler. Samples were prepared via microwave digestion using a Milestone Ultrawave Single Reaction Chamber Microwave Digester with 15 vial rack. Approximately 0.25 g of sample was digested in 4 mL of concentrated nitric acid, 1 mL H₂O, and 1 mL fluoroboric acid. The microwave digester was set to ramp at 10° C./min to 250° C. and hold at 250° C. for 20 min. The pressure was maintained at a max pressure of 100 bar. After digestion, the samples were diluted to 50 mL using DI water. Quantitative analysis of manganese was performed by measuring 257.61 nm wavelength in axial mode. Axial mode was set to an RF Power of 1.4 kW, argon flow rate 13 L/min, auxiliary 1 L/min, nebulizer 0.6 L/min, uptake delays of 20 sec, max rinse time of 120 sec with intelligent rinse enabled. Calibration standards were measured using a six-point calibration curve (0.1 ppm-20 ppm). The manganese calibration curve had a correlation coefficient of 0.9998. Quality control includes running blanks and calibration standards before and after manganese analysis.

### ICP-MS

Bromine concentration was measured with ICP-MS. 10 mg of vacuum distilled benzoic acid was dissolved in 10 mL of DI H₂O. The solution was further diluted in a ratio of 10:1 in a solution of milliQ water containing 1 wt % triethanolamine and analyzed using an Agilent 7700 ICP-MS using a five-point calibration curve from a bromine standard purchased from High Purity Standards. Calibration curve for Br resulted in a correlation coefficient value of 0.9999, a detection limit of 35 ppt, and a background level of 6.3 ppb. Blanks and calibration standards were run two times both before and after full quantification.

### Elemental Analysis for C, H, N

The post-consumer PS samples were analyzed for carbon, hydrogen, and nitrogen using a LECO CHN 628 (LECO Corporation). The analysis parameters are a combustion temperature of 950° C. and an afterburner temperature of 850° C. The burn profile is as follows: step 1—high furnace flow for 40 sec; step 2—medium furnace flow for 30 sec; and step 3—high furnace flow for 30 sec. The ballast has an equilibration time of 30 sec with a 300 sec not filled timeout. The aliquot loop has a fill pressure drop of 200 mmHg with an equilibration time of 8 sec. A sample size of 100 mg was wrapped into a tin cup prior to analysis. This method was calibrated with EDTA (Part #—502-896—LECO Corporation).

### Elemental Analysis for Br, Cl

Total bromine and chlorine were determined using combustion ion chromatography (CIC). The combination of an Agilent 1100 Series HPLC, Thermo Scientific DRS Suppressor, and Column Dionex ADRS600 4 mm CMD are used. About 20 mg was weighed accurately into an aluminum sample boat and wrapped in ashless filter paper. Then, the sample was combusted in an oxygen combustion flask and filtered, if necessary, prior to analysis. The sample solutions were analyzed by ion chromatography using a polymeric anion exchange resin and a sodium carbonate/sodium hydrogen carbonate mobile phase. The sample solution was injected into the ion exchange columns using a liquid pump and liquid autosampler. Ions were detected as they eluted from the end of the column by a conductivity detector. Anions in the sample were identified and quantified by comparison to diluted standard solutions prepared from NIST traceable standard solutions. System suitability and standard confirmation were demonstrated from replicate injections of a working standard solution and a check standard solution prior to injections of the sample solutions. The working standard solution was also injected and evaluated periodically throughout the sequence and at the end of the sequence to ensure that the system suitability was maintained. The response from the sample solution was converted to concentration in solution and corrected from any response observed in blank preparations. The concentration in solution was converted to concentration in the sample by taking into account the sample amount and the dilution volume.

### Measurement of Temperature of Liquid Benzoic Acid

To measure the liquid phase temperature at a desired gas-phase temperature, a Parr pressure vessel was modified by replacing the plug with a bored through union (Swagelok SS-200-1-2BT). A K-type thermocouple (Omega KQXL-18G-12) was placed through the fitting with the end submerged in the liquid phase benzoic acid. In the reactor, benzoic acid 3.0 g was added, and charged with the mixture of O₂ at 7 bar and N₂ at 53 bar. The temperature was monitored by connecting the thermocouple to a Watlow temperature controller.

### Process Modeling and Techno-Economic Analysis

Conceptual process models for the conversion of PS to benzoic acid were developed using Aspen Plus V14. The oxidation of PS to benzoic acid was modeled using the ENRTL-HG and ELECNRTL property methods based on experimental results from and literature. The conversion of benzoic acid to adipic acid was adapted from the model previously described which uses the NRTL property method.

### Life Cycle Assessment

To determine the environmental impacts of the conversion of post-consumer polystyrene to adipic acid, a cradle-to-gate life cycle assessment was conducted using Brightway2 software, version 2.11. All co-products were treated using system expansion (e.g. assuming co-production of excess steam offsets traditional steam production). Background data are derived from Ecoinvent v3.9.1, and foreground data are derived from the material and energy balances provided by the process flow models developed herein using Aspen Plus. Life cycle stages include the collection and compaction of post-consumer PS waste, oxidation to benzoic acid, bioconversion to muconic acid, and final conversion to adipic acid. The ReCiPe 2016 midpoint H impact assessment method was utilized to investigate all environmental impacts, except for global warming potential, which was measured using the Intergovernmental Panel on Climate Change (IPCC) 2021 method. For the sensitivity analysis, a Monte Carlo analysis was performed with 1,000 iterations, providing standard deviations for each environmental impact category.

The foregoing discussion and examples have been presented for purposes of illustration and description. The foregoing is not intended to limit the aspects, embodiments, or configurations to the form or forms disclosed herein. In the foregoing Detailed Description for example, various features of the aspects, embodiments, or configurations are grouped together in one or more embodiments, configurations, or aspects for the purpose of streamlining the disclosure. The features of the aspects, embodiments, or configurations, may be combined in alternate aspects, embodiments, or configurations other than those discussed above. This method of disclosure is not to be interpreted as reflecting an intention that the aspects, embodiments, or configurations require more features than are expressly recited in each claim. Rather, as the following claims reflect, inventive aspects lie in less than all features of a single foregoing disclosed embodiment, configuration, or aspect. While certain aspects of conventional technology have been discussed to facilitate disclosure of some embodiments of the present invention, the Applicants in no way disclaim these technical aspects, and it is contemplated that the claimed invention may encompass one or more of the conventional technical aspects discussed herein. The following claims are hereby incorporated into this Detailed Description, with each claim standing on its own as a separate aspect, embodiment, or configuration.

We claim:

1. A method for converting polystyrene to a compound of interest using an autoxidation reaction comprising reacting polystyrene with catalysts comprising manganese and bromide to obtain benzoic acid and further comprising the steps of reacting the obtained benzoic acid to obtain the compound of interest.

2. The method of claim 1 wherein polystyrene autoxidation showed that yields higher than 85% can be achieved with the presence of a Mn(OAc)₂ and NaBr catalysts.

3. The method of claim 1 wherein the vacuum distillation of the products resulted in high-purity benzoic acid, which can be further bioprocessed into muconic acid.

4. The method of claim 3 wherein hydrogenation of muconic acid using Mn(OAc)₂ and NaBr catalysts results in adipic acid.

5. The method of claim 1 wherein polystyrene autoxidation takes place in benzoic acid as a solvent.

6. The method of claim 3 wherein the further comprising converting the generated muconic acid into a composition of matter selected from the group consisting of adipic acid, terephthalic acid, adiponitrile, acrylic acid and caprolactam.

7. A method for making adipic acid from polystyrene comprising autoxidation of polystyrene in a solution of benzoic acid to generate benzoic acid wherein the benzoic acid is metabolized by a non-naturally occurring microbe to make muconic acid; and wherein the muconic acid is hydrogenated to make adipic acid.

8. The method of claim 7 wherein the autoxidation of polystyrene to benzoic acid occurs in a solution comprising oxygen, manganese and bromide.

9. The method of claim 8 wherein the manganese is loaded into the solution as Mn(OAc)₂ at about 7.1 wt % and wherein the bromide is added into the solution as NaBr at 4.0 wt % resulting in a yield of benzoic acid up to 78% of the starting polystyrene.

10. The method of claim 9 wherein the polystyrene is loaded in a range of from 0.04 g polystyrene per gram of benzoic acid up to about 0.45 g polystyrene per gram of benzoic acid.

11. The method of claim 8 wherein the yield of benzoic acid is from 88% to 94% of starting polystyrene and wherein the bromide is added into the solution as NaBr at 2.0 wt %.

12. The method of claim 11 wherein the reaction is at about 165 degrees Celsius.

13. The method of claim 11 wherein the reaction occurs up to 2 hours.

14. The method of claim 11 wherein the reaction occurs up to 4 hours.

15. The method of claim 7 wherein the polystyrene is expanded polystyrene.

16. The method of claim 7 wherein the non-naturally occurring microbe is Pseudomonas and comprises a deletion of catB.

17. The method of claim 16 wherein the non-naturally occurring microbe further comprises a gene encoding for a catechol 1,2-dioxygenase.

18. The method of claim 17 wherein muconic acid is isolated and hydrogenated to adipic acid.

19. The method of claim 12 wherein the non-naturally occurring microbe comprises Pseudomonas putida.

20. The method of claim 12 wherein the non-naturally occurring microbe comprises Pseudomonas putida KT2440-CJ074.
